(19) United States
(12) Patent Application Publication
(10) Pub. No.: US 2025/0171535 A1
(43) Pub. Date: May 29, 2025

(54) COMPOSITIONS AND METHODS FOR DEPLETION OF DISEASED HEMATOPOIETIC STEM CELLS

(71) Applicant: Jasper Therapeutics, Inc., Redwood City, CA (US)

(72) Inventors: Wendy PANG, Palo Alto, CA (US); Hye-Sook KWON, San Mateo, CA (US)

(21) Appl. No.: 18/841,051

(22) PCT Filed: Feb. 28, 2023
(86) PCT No.: PCT/US2023/014175

§ 371 Date: Aug. 23, 2024

Related U.S. Application Data
(60) Provisional application No. 63/314,917, filed on Feb. 28, 2022.

(51) Int. Cl. C07K 16/28; A61K 9/00; A61K 31/45; A61K 31/706; A61K 38/17; A61K 39/00; A61P 35/02

(52) U.S. Cl. CPC C07K 16/2803; A61K 9/0019; A61K 31/45; A61K 31/706; A61K 38/179; A61P 35/02; A61K 2039/505; A61K 2039/54; A61K 2039/545; C07K 2317/24; C07K 2317/565; C07K 2317/76

(57) ABSTRACT

Provided herein are compositions and methods related to depletion of diseased hematopoietic stem cells (HSC) using an anti-c-kit antibody. The compositions and methods described herein may be used to treat a subject in need of diseased HSC depletion due to a variety of diseases or disorders, such as myelodysplastic syndrome and acute myeloid leukemia.

### RELATED APPLICATIONS

This application claims priority to U.S. Provisional Patent Application No. 63/314,917, filed Feb. 28, 2022, incorporated herein by reference in its entirety.

### REFERENCE TO AN ELECTRONIC SEQUENCE LISTING

The contents of sequence the electronic listing (JATH_008_01WO_SeqList_ST26.xml; Size: 43,722 bytes; and Date of Creation: Feb. 28, 2023) is herein incorporated by reference in its entirety.

### FIELD OF THE INVENTION

The present disclosure relates to compositions and methods for depletion of hematopoietic stem cells (HSC). The compositions and methods described herein may be used to treat patients requiring diseased HSC depletion for a variety of different diseases or disorders, including but not limited to myelodysplastic syndrome (MDS) and acute myeloid leukemia (AML).

### BACKGROUND

Myelodysplastic syndrome (MDS) is a hematologic malignancy characterized by the presence of defective hematopoietic stem cells (HSC). Current treatments for MDS include hematopoietic stimulating agents for temporary resolution of associated symptoms. These may include chronic transfusions, erythropoietin stimulating agents for red blood production, or various chemotherapies. However, there are no existing treatments that specifically target diseased HSC relative to healthy HSC. Therefore, there is a need for innovative agents and regimens that selectively target diseased HSC and permit healthy HSC to undergo normal hematopoiesis in the bone marrow. The present disclosure meets this need by providing agents and regimens for selective depletion of diseased HSC in MDS, acute myeloid leukemia (AML), and other disorders.

### BRIEF SUMMARY OF THE INVENTION

The present disclosure provides inter alia a method of depleting diseased hematopoietic stem cells (HSC) relative to healthy HSC by administering anti-c-kit antibodies to a patient. In particular embodiments, the diseased HSC are selectively depleted as compared to healthy HSC.

In one aspect, the disclosure provides a method of depleting endogenous diseased hematopoietic stem cells (HSCs) in a subject, comprising administering to the subject an anti-c-kit antibody, wherein the method results in an increase in the percentage of healthy hematopoietic stem cells (HSCs) present in the subject's bone marrow, e.g., immediately following the conclusion of treatment, or at about one day, two days, three days, four days, five days, six days, ten days, one week, two weeks, one month, two months, four months, or six months following the conclusion of treatment. In particular embodiments, the diseased HSC are selectively depleted. In particular embodiments, the anti-c-kit antibody comprises one or more complementarity-determining regions (CDRs) present in a monoclonal antibody selected from the group consisting of: SR-1, JSP191, MGTA-117, FSI-174, CDX0158, CDX0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112, optionally JSP191, CDX0158, CDX0159, or FSI-174. In particular embodiments, the anti-c-kit antibody comprises one or more complementarity-determining regions (CDRs) present in a humanized version of a monoclonal antibody selected from the group consisting of: ACK2, ACK4, 2B8, 3C11, MR-1, and CD122. In certain embodiments, the anti-c-kit antibody comprises the CDRs of an antibody that blocks the binding of stem cell factor (SCF) to stem cell factor receptor (CD117), optionally wherein the antibody is JSP191.

In particular embodiments, the subject is administered about 0.01 mg/kg to about 20 mg/kg of the anti-c-kit antibody, optionally about 0.1 mg/kg to about 10 mg/kg of the anti-c-kit antibody. In particular embodiments of any of the methods disclosed herein, the subject is administered about 0.6 mg/kg of the anti-c-kit antibody. In particular embodiments, the subject is administered about 2 mg/kg of the anti-c-kit antibody. In particular embodiments, the anti-c-kit antibody is delivered to the subject systemically, optionally wherein the anti-c-kit antibody is delivered intravenously or subcutaneously. In particular embodiments, the anti-c-kit antibody is delivered to the subject locally, optionally wherein the anti-c-kit antibody is delivered directly to the bone marrow. In particular embodiments, the subject is administered two or more doses of the anti-c-kit antibody. In particular embodiments, one of the two or more doses is administered about every week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, or about every 12 weeks, optionally wherein the time interval between each dose varies. In particular embodiments, the subject's bone marrow comprises at least 1%, at least 2%, at least 3%, at least 4%, at least 5%, at least 6%, at least 7%, at least 8%, at least 9%, at least 10%, at least 15%, or at least 20% healthy HSCs prior to treatment.

In particular embodiments of any of the methods disclosed herein, the method is used to treat a disease or disorder selected from the group consisting of: acute myeloid leukemia (AML), chronic myeloid leukemia (CML), chronic myelomonocytic leukemia (CMML), acute lymphoblastic leukemia (ALL), hodgkin lymphoma, non-hodgkin lymphoma, clonal hematopoiesis of indeterminate potential (CHIP), clonal cytopenia of undetermined significance (CCUS) myelodysplastic syndromes (MDS), idiopathic cytopenia of undetermined significance (ICUS), and myeloproliferative neoplasms (MPN). In particular embodiments, the disease or disorder is an MDS. In particular embodiments, the MDS is a lower risk MDS. In particular embodiments, the subject has failed an erythropoiesis-stimulating agent (ESA) therapy, optionally wherein the ESA therapy comprises Erythropoietin and/or Darbepoetin. In particular embodiments, the method has reduced side effects as compared to treatment with a chemotherapeutic agent, optionally wherein the chemotherapeutic agent comprises Azacytidine, Lenalidomide, Decitabine, and/or Luspatercept. In particular embodiments, the reduced side effects comprise lower risk of hematologic toxicity and lower risk of mortality by infection. In particular embodiments, the method results in an increase in the percentage of normal HSCs in the bone marrow to at least 50%, at least 60%, at least 70%, at least 80%, at least 90%, at least 95%, at least 99%, or at least 100% of all HSCs, and optionally where the method results in long term dominance of healthy hematopoietic stem cells (HSCs) in the subject's bone marrow. In particular embodiments, the subject does not need or have a hematopoietic cell transplant (HCT) within at least 4 months, at least 6 months, at least 1 year, or at least 2 years following administration of the anti-c-kit antibody.

In another aspect, the disclosure provides a method of treating a disease or disorder, optionally myelodysplastic syndrome (MDS) or acute myeloid leukemia (AML), in a subject, the method comprising administering to the subject an anti-c-kit antibody in a dose effective to achieve long term dominance of healthy hematopoietic stem cells (HSCs) in the subject's bone marrow. In particular embodiments, the disease or disorder is myelodysplastic syndrome (MDS). In particular embodiments, the MDS is a lower risk MDS. In particular embodiments, the disease or disorder is acute myeloid leukemia (AML). In particular embodiments, the subject has failed an erythropoiesis-stimulating agent (ESA) therapy, optionally wherein the ESA therapy comprises Erythropoietin and/or Darbepoetin. In particular embodiments, the method selectively depletes diseased HSCs as compared to healthy HSCs. In particular embodiments, the anti-c-kit antibody comprises one or more complementarity-determining regions (CDRs) present in a monoclonal antibody selected from the group consisting of: SR-1, JSP191, MGTA-117, FSI-174, CDX-0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112. In particular embodiments, the anti-c-kit antibody comprises one or more complementarity-determining regions (CDRs) present in a humanized version of a monoclonal antibody selected from the group consisting of: ACK2, ACK4, 2B8, 3C11, MR-1, and CD122. In particular embodiments, the anti-c-kit antibody comprises the CDRs of an antibody that blocks the binding of stem cell factor (SCF) to stem cell factor receptor (CD117), optionally wherein the antibody is JSP191.

In particular embodiments of methods disclosed herein, the subject is administered about 0.01 mg/kg to about 10 mg/kg of the anti-c-kit antibody, optionally about 0.1 mg/kg to about 10 mg/kg of the anti-c-kit antibody. In particular embodiments, the subject is administered about 0.6 mg/kg of the anti-c-kit antibody. In particular embodiments, the subject is administered about 2 mg/kg of the anti-c-kit antibody. In particular embodiments, the anti-c-kit antibody is delivered to the subject systemically, optionally wherein the anti-c-kit antibody is delivered intravenously or subcutaneously. In particular embodiments, the anti-c-kit antibody is delivered to the subject locally, optionally wherein the anti-c-kit antibody is delivered directly to the bone marrow. In particular embodiments, the subject is administered two or more doses of the anti-c-kit antibody. In particular embodiments, one of the two or more doses is administered every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks, optionally wherein the time interval between dosages varies. In particular embodiments, the subject's bone marrow comprises at least 1%, at least 2%, at least 3%, at least 4%, at least 5%, at least 6%, at least 7%, at least 8%, at least 9%, at least 10%, at least 15%, or at least 20% healthy HSCs prior to treatment.

In particular embodiments, the methods disclosed herein have reduced side effects as compared to treatment with a chemotherapeutic agent, optionally wherein the chemotherapeutic agent comprises Azacytidine, Lenalidomide, Decitabine, and/or Luspatercept. In particular embodiments, the reduced side effects are lower risk of hematologic toxicity and lower risk of mortality by infection. In particular embodiments, the method results in an increase in the percentage of normal HSCs in the bone marrow to at least 50%, at least 60%, at least 70%, at least 80%, at least 90%, at least 95%, at least 99%, or at least 100% of all HSCs. In particular embodiments, the subject does not have a hematopoietic cell transplant (HCT) within at least 4 months, at least 6 months, at least 1 year, or at least 2 years following administration of the anti-c-kit antibody. In particular embodiments, the anti-c-kit antibody, e.g., JSP191, is administered in combination with an anti-CD47 antibody, e.g., for treatment of AML. In particular embodiments, the anti-c-kit antibody is administered in combination with a chemotherapeutic agent. In particular embodiments, the chemotherapeutic agent is azacytidine, e.g., for treatment of MDS.

### BRIEF DESCRIPTION OF THE DRAWINGS

FIG. 1 is a schematic diagram of the effect of administering an illustrative anti-c-kit antibody, e.g., JSP191, on the hematopoiesis of normal and diseased cells in a patient with a hematologic malignancy, e.g., myelodysplastic syndrome (MDS) or acute myeloid leukemia (AML).

FIG. 2 is a bar graph of the percent (%) depletion of CD34+CD45RA-CD117+ cells in nine myelodysplastic syndrome (MDS) and acute myeloid leukemia (AML) patients treated with a single dose of anti-c-kit antibody, measured 10-14 days post-treatment.

FIG. 3 is a flow diagram demonstrating an example target patient population, e.g., lower risk myelodysplastic syndrome (MDS) patients, for anti-c-kit antibody treatment to deplete diseased hematopoietic stem cells (HSC).

### DETAILED DESCRIPTION OF THE INVENTION

The present disclosure provides treatments for myelodysplastic syndrome (MDS) MDS, acute myeloid leukemia (AML), and other patients with anti-c-kit antibodies in a dose effective to deplete diseased hematopoietic stem cells (HSC) and increase the percentage of healthy HSC in the bone marrow. In particular embodiments, the diseased HSC are selectively depleted. In certain embodiments, the anti-c-kit antibody treatments described herein reduce diseased HSC to a greater degree than healthy HSC are reduced, thus leaving healthy cells to re-establish dominance in the bone marrow, and thereby increasing the number of healthy HSCs and reducing the need for hematopoietic stem cell transplant (HCT), total body irradiation (TBI), and/or chemotherapies. In particular embodiments, the methods achieve long term dominance of healthy hematopoietic stem cells (HSCs) in the subject's bone marrow, e.g., for at least six months, at least one year, at least two years, at least five years, or at least 10 years.

The treatments described herein have been shown to be effective in treating myelodysplastic syndrome (MDS) and acute myeloid leukemia (AML) (see Examples). However, these treatments are not intended to be limiting to these disease populations alone. Compositions and methods disclosed herein may be used to treat other disorders for which selective depletion of diseased HSC is indicated. Relevant diseases or disorders include but are not limited to: acute myeloid leukemia (AML), chronic myeloid leukemia (CML), chronic myelomonocytic leukemia (CMML), acute lymphoblastic leukemia (ALL), hodgkin lymphoma, non-hodgkin lymphoma, clonal hematopoiesis of indeterminate potential (CHIP), clonal cytopenia of undetermined significance (CCUS) myelodysplastic syndromes (MDS), idiopathic cytopenia of undetermined significance (ICUS), and myeloproliferative neoplasms (MPN).

It is to be understood that this invention is not limited to the particular methodology, products, apparatus and factors described, as such methods, apparatus and formulations may, of course, vary. It is also to be understood that the terminology used herein is for the purpose of describing particular embodiments only, and it is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

As used herein, “antibody” includes reference to an immunoglobulin molecule immunologically reactive with a particular antigen, and includes both polyclonal and monoclonal antibodies. The term also includes genetically engineered forms such as humanized antibodies, chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Selection of antibodies for endogenous stem cell ablation may be based on a variety of criteria, including selectivity, affinity, cytotoxicity, etc. The phrase “specifically (or selectively) binds” to an antibody, when referring to a protein or peptide, refers to a binding reaction that is determinative of the presence of the protein, in a heterogeneous population of proteins and/or other biologics. Thus, under designated immunoassay conditions, the specified antibodies bind to a particular protein sequence at least two times the background binding and more typically more than 10 to 100 times background binding.

Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into a Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

The selectivity of a particular antibody is typically determined by the ability of one antibody to competitively inhibit binding of the second antibody to the antigen, or by the ability of an antibody to cross-react with multiple epitopes. Any of a number of competitive binding assays can be used to measure competition between two antibodies to the same antigen, or between two antigens to one antibody. An exemplary assay is a BIACORE™ assay. Briefly in these assays, binding sites can be mapped in structural terms by testing the ability of interactants, e.g. different antibodies, to inhibit the binding of another. Injecting two consecutive antibody or antigen samples in sufficient concentration can identify pairs of competing antibodies for the same binding epitope. The antibody samples should have the potential to reach a significant saturation with each injection. The net binding of the second antibody injection is indicative for binding epitope analysis. Two response levels can be used to describe the boundaries of perfect competition versus non-competing binding due to distinct epitopes. The relative amount of binding response of the second antibody injection relative to the binding of identical and distinct binding epitopes determines the degree of epitope overlap.

The term “polynucleotide” refers to a polymeric form of nucleotides of any length, including deoxyribonucleotides or ribonucleotides, or analogs or mixtures thereof. A polynucleotide may comprise modified nucleotides, such as methylated nucleotides and nucleotide or nucleoside analogs, and may be interrupted by non-nucleotide components. If present, modifications to the nucleotide structure may be imparted before or after assembly of the polymer. The term polynucleotide, as used herein, includes, but is not limited to, double- and single-stranded molecules, and mixtures thereof. Unless otherwise specified or required, any embodiment of the invention described herein that is a polynucleotide encompasses both the double-stranded form and each of two complementary single-stranded forms known or predicted to make up the double-stranded form, whether as RNA or DNA, or a mixture thereof.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the terms “identity” and “identical” refer, with respect to a polypeptide or polynucleotide sequence-of-interest, to the percentage of exact matching residues in an alignment of that the sequence-of-interest to a reference sequence, such as an alignment generated by the BLAST algorithm. Identity is calculated, unless specified otherwise, across the full length of the reference sequence. Thus a sequence-of-interest “shares at least x % identity to” a reference sequence if, when the reference sequence is aligned (as a query sequence) is aligned to the sequence-of-interest (as subject sequence), at least x % (rounded down) of the residues in the subject sequence are aligned as an exact match to a corresponding residue in the query sequence, the denominator being the full length of the reference sequence plus the lengths of any gaps inserted into the reference sequence by alignment of the reference sequence to the sequence-of-interest. Where the subject sequence has variable positions (e.g., residues denoted X), an alignment to any residue in the query sequence is counted as a match. Sequence similarity (i.e., identity) can be determined in a number of different manners. To determine sequence identity, sequences can be aligned using the methods and computer programs, including BLAST, available over the worldwide web at ncbi.nlm.nih.gov/BLAST/. Sequence alignments may be performed using the NCBI Blast service (BLAST+ version 2.12.0) or another program giving the same results. Unless indicated to the contrary, sequence identity is determined using the BLAST algorithm (e.g., bl2seq) with default parameters.

The term “native” or “wild-type” as used herein refers to a nucleotide sequence, e.g., gene, or gene product, e.g., RNA or polypeptide, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 50% or more, 60% or more, or 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

The term “stem cell” as used herein refers to a mammalian cell that has the ability both to self-renew, and to generate differentiated progeny (see Morrison et al. (1997) Cell 88:287-298). Endogenous stem cells may be characterized by the presence of markers associated with specific epitopes. The term “hematopoietic stem cells (HSC)” as used herein refers to multipotent cells that reside in the bone marrow (BM) and are responsible for the life-long production of mature blood cells.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered before or during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, the term “substantially” means by a significant or large amount or degree. For example, to “substantially” increase may mean to increase by at least two-fold, at least three-fold, at least four-fold, at least five-fold, or at least ten-fold, and to “substantially” decrease may mean to decrease by at least 30%, at least 40%, at least 50%, at least 60%, at least 70%, at least 80%, or at least 90% In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### I. Depletion of Diseased HSCS

In some embodiments, the treatments described herein deplete diseased hematopoietic stem cells (HSC) from the bone marrow of a subject. In some embodiments, “a diseased HSC” is an HSC that does not differentiate or has a significantly reduced differentiation capacity, does not divide or has a significantly reduced capacity for cell division, fails to produce functional blood cells or produces a significantly reduced amount of functional blood cells, and/or is otherwise classified as a hematological malignancy. In certain embodiments, a significant difference is a statistically significant difference, having a p-value of 5% or lower. In some embodiments, the disclosure provides methods for selectively depleting endogenous diseased hematopoietic stem cells (HSCs) in a subject, and/or methods that result in an increase in the relative percentage of healthy HSC, by administering an anti-c-kit antibody.

In some embodiments, the treatments described herein deplete diseased HSC in a subject. In some embodiments, depleting diseased HSC refers to depletion of diseased HSC in an amount of about 2%, about 5%, about 10%, about 20%, about 30%, about 40%, about 50%, about 60%, about 70%, about 80%, about 90%, about 95%, about 98%, about 99%, or about 100% of total diseased HSC in a subject.

In some embodiments, the treatments described herein selectively deplete diseased HSC in a subject. In particular embodiments, selective depletion of diseased HSCs according to a disclosed method results in the depletion of a greater number or percentage of diseased HSCs as compared to the number or percentage of non-diseased or healthy HSCs that are depleted.

In some embodiments, the treatments described herein result in an increase in the relative percentage of healthy HSC in a subject. In some embodiments, the treatments described herein increase the relative percentage of healthy HSC (relative to total HSC) in a subject by about 50%, about 60%, about 60%, about 70%, about 80%, about 90%, about 100%, about 200%, about 300%, about 400%, about 500%, about 600%, about 700%, about 800%, about 900%, or about 1000%.

### Anti-C-Kit Antibodies

In some embodiments, the disclosure provides methods for selectively depleting endogenous diseased hematopoietic stem cells (HSCs) in a subject and/or increasing the relative percentage of healthy HSC, by administering an anti-c-kit antibody. In some embodiments, the compositions and methods disclosed herein may be applicable to any anti-c-kit antibody that blocks stem cell factor (SCF) binding to a c-kit/CD117 protein of an endogenous HSC. In some embodiments the antibody is a monoclonal anti-human c-kit antibody. In some embodiments, the antibody is an antigen-binding fragment of an antibody that blocks stem cell factor (SCF) binding.

A number of antibodies contemplated by the disclosure that specifically bind human CD117 are known in the art and/or commercially available, including without limitation JSP-191, SR1, 2B8, ACK2, YB5-B8, 57A5, and 104D2. In certain embodiments, the anti-c-kit antibody is selected from the group consisting of: JSP191 (Jasper Therapeutics; Redwood City, CA); CDX-0158 (formerly KTN0158) or CDX-0159 (Celldex Therapeutics, Hampton, NJ); MGTA-117 (AB85) (Magenta Therapeutics, Cambridge, MA); CK6 (Magenta Therapeutics, Cambridge, MA); AB249 (Magenta Therapeutics, Cambridge, MA); and FSI-174 (Gilead, Foster City, CA). Antibodies from Magenta Therapeutics contemplated by the disclosure include but are not limited to those that are disclosed in US Patent Application Publication No. 20190153114, PCT Application Publication Nos. WO2019084064, WO2020/219748, and WO2020/219770. The FSI-174 antibody is disclosed in PCT application Publication No. WO2020/112687 and U.S. Patent Application Publication No. 20200165337. The disclosure includes but is not limited to any anti-c-kit antibodies and/or CDR sets disclosed in any of the patent application disclosed herein, which are all incorporated by reference in their entireties.

In certain embodiments, the anti-c-kit antibody binds to the extracellular region of CD117, i.e., amino acids 26-524. The sequence of this region is shown below:

(SEQ ID NO: 1)
QPSVSPGEPSPPSIHPGKSDLIVRVGDEIRLLCTDPGFVKWTFEILDET
NENKQNEWITEKAEATNTGKYTCTNKHGLSNSIYVFVRDPAKLFLVDRS
LYGKEDNDTLVRCPLTDPEVTNYSLKGCQGKPLPKDLRFIPDPKAGIMI
KSVKRAYHRLCLHCSVDQEGKSVLSEKFILKVRPAFKAVPVVSVSKASY
LLREGEEFTVTCTIKDVSSSVYSTWKRENSQTKLQEKYNSWHHGDFNYE
RQATLTISSARVNDSGVFMCYANNTFGSANVTTTLEVVDKGFINIFPMI
NTTVFVNDGENVDLIVEYEAFPKPEHQQWIYMNRTFTDKWEDYPKSENE
SNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSG
PPFGKLVVQSSIDSSAFKHNGTVECKAYNDVGKTSAYFNFAFKGNNKEQ
IHPHTLFTP.

Illustrative anti-c-kit antibodies include, but are not limited to, SR-1, JSP191, 8D7, K45, 104D2, CK6, YB5.B8, AF-2-1, AF11, AF12, AF112, AF-3, AF-1-1, NF, NF-2-1, NF11, NF12, NF112, NF-3, HF11, HF12, and HF112. A number of antibodies contemplated by the disclosure that specifically bind human CD117 are commercially available, including without limitation SR1, 2B8, ACK2, YB5-B8, 57A5, 104D2, etc. In certain embodiments, the anti-c-kit antibody is selected from the group consisting of: JSP191, CDX-0159 (from Celldex Therapeutics, Hampton, NJ), MGTA-117 (AB85) (from Magenta Therapeutics, Cambridge, MA), CK6 (from Magenta Therapeutics, Cambridge, MA), AB249 (from Magenta Therapeutics, Cambridge, MA), and FSI-174 (from Gilead, South San Francisco, CA). The antibodies from Magenta Therapeutics are disclosed in US Patent Application Publication No. 20190153114. In certain embodiments, the antibody is one disclosed in any of U.S. Pat. Nos. 7,915,391, 8,436,150, or 8,791,249. In certain embodiments, the antibody is one disclosed in U.S. Pat. Application Publ. No. 20200165337 or any of PCT Publication Nos. WO 2020/112687, WO2020/219748, WO 2020/219770, or WO 2019/084064.

In particular embodiments, the antibody is a humanized form of SRI, a murine anti-c-kit antibody described in U.S. Pat. Nos. 5,919,911 and 5,489,516. The humanized form, JSP191, is disclosed in U.S. Pat. Nos. 7,915,391, 8,436,150, and 8,791,249. JSP191 is an aglycosylated IgG1 humanized antibody. JSP191 specifically binds to human CD117, a receptor for stem cell factor (SCF), which is expressed on the surface of hematopoietic stem and progenitor cells. JSP191 blocks SCF from binding to CD117 and disrupts stem cell factor (SCF) signaling, leading to the depletion of hematopoietic stem cells. JSP191 is a heterotetramer consisting of 2 heavy chains of the IgG1 subclass and 2 light chains of the kappa subclass, which are covalently linked through disulfide bonds. There are no N-linked glycans on JSP191 due to an intentional substitution from an asparagine to glutamine at heavy chain residue 297. The sequences of the heavy chains and light chains of JSP191 are disclosed as SEQ ID NO: 4 from U.S. Pat. No. 8,436,150 and SEQ ID NO: 2 from U.S. Pat. No. 8,436,150, respectively.

The sequences of the heavy chains and light chains of JSP191 are disclosed as SEQ ID NO: 4 from U.S. Pat. No. 8,436,150 and SEQ ID NO: 2 from U.S. Pat. No. 8,436,150, respectively. The sequences of the heavy and light chains of JSP191 are:

Heavy Chain:
(SEQ ID NO: 2)
MDWTWRVFCLLAVAPGAHSQVQLVQSGAEVKKPGASVKVSCKASGYTFT
SYNMHWVRQAPGQGLEWMGVIYSGNGDTSYNQKFKGRVTITADKSTSTA
YMELSSLRSEDTAVYYCARERDTRFGNWGQGTLVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKF
NWYVDGVEVHNAKTKPREEQYQSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPGK
and
Light Chain:
(SEQ ID NO: 3)
MVLQTQVFISLLLWISGAYGDIVMTQSPDSLAVSLGERATINCRASESV
DIYGNSFMHWYQQKPGQPPKLLIYLASNLESGVPDRFSGSGSGTDFTLT
ISSLQAEDVAVYYCQQNNEDPYTFGGGTKVEIKRTVAAPSVFIFPPSDE
QLKSGTASVVCLLNNFYPREAKVQWKVDNALQSGNSQESVTEQDSKDST
YSLSSTLTLSKADYEKHKVYACEVTHQGLSSPVTKSFNRGEC

In certain embodiments, the variable heavy domain of JSP191 comprises the following sequence:

(SEQ ID NO: 4)
QVQLVQSGAEVKKPGASVKVSCKASGYTFTSYNMHWVRQAPGQGLEWMG
VIYSGNGDTSYNQKFKGRVTITADKSTSTAYMELSSLRSEDTAVYYCAR
ERDTRFGNWGQGTLVTVSS

In certain embodiments, the variable light chain domain of JSP191 comprises the following sequence:

(SEQ ID NO: 5)
DIVMTQSPDSLAVSLGERATINCRASESVDIYGNSFMHWYQQKPGQPPK
LLIYLASNLESGVPDRFSGSGSGTDFTLTISSLQAEDVAVYYCQQNNED
PYTFGGGTKVEIK.

The CDRs present in JSP191 are as follows: VH CDR1=YNMH (SEQ ID NO: 6); VH CDR2=IYSGNGDTSYNQKFKG (SEQ ID NO: 7); VH CDR3=ERDTREGN (SEQ ID NO: 8); VL CDR1=RASESVDIYGNSFMH (SEQ ID NO: 9); VL CDR2=LASNLES (SEQ ID NO: 10); and VL CDR3=QQNNEDPYT (SEQ ID NO: 11).

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization. CDX-0159 and other anti-c-kit antibodies are described in U.S. Pat. No. 10,781,267, and in particular embodiments, an anti-c-kit disclosed herein comprises the CDRs of any of the antibodies disclosed therein. In certain embodiments, the anti-c-kit antibody comprises: (i) a light chain variable region (“VL”) comprising the amino acid sequence: DIVMTQSPSX_{K1}LSASVGDRVTITCKASQNVRTNVAWYQQKPGKAPKX_{K2}LIYSASYR YSGVPDRFX_{K3}GSGSGTDFTLTISSLQX_{K4}EDFAX_{K5}YX_{K6}CQQYNSYPRTFGGGTKVEIK (SEQ ID NO: 12), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain, and X_{K6} is an amino acid with an aromatic side chain; and (ii) a heavy chain variable region (“VH”) comprising the amino acid sequence: QVQLVQSGAEX_{H1}KKPGASVKX_{H2}SCKASGYTFTDYYINAVVX_{H3}QAPGKGLEWIARI YPGSGNTYYNEKFKGRX_{H4}TX_{H5}TAX_{H6}KSTSTAYMX_{H7}LSSLRSEDX_{H8}AVYFCARGV YYFDYWGQGTTVTVSS (SEQ ID NO: 13), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In specific aspects, described herein are antibodies (e.g., human or humanized antibodies), including antigen-binding fragments thereof, comprising: (i) VH CDRs of a VH domain comprising the amino acid sequence

(SEQ ID NO: 14)
QVQLKQSGAELVRPGASVKLSCKASGYTFTDYYINWVKQRPGQGLEWIA
RIYPGSGNTYYNEKFKGKATLTAEKSSSTAYMQLSSLTSEDSAVYFCAR
GVYYFDYWGQGTTLTVSS
or
(SEQ ID NO: 15)
QVQLKQSGAELVRPGASVKLSCKASGYTFTDYYINWVKQRPGQGLEWIA
RIYPGSGNTYYNEKFKGKATLTAEKSSSTAYMQLSSLTSEDSAVYFCAR
GVYYFDYWGQGTTLTVSA,
and

(ii) VL CDRs of a VL domain comprising the amino acid sequence

(SEQ ID NO: 16)
DIVMTQSQKFMSTSVGDRVSVTCKASQNVRTNVAWYQQKPGQSPKALIY
SASYRYSGVPDRFTGSGSGTDFTLTISNVQSEDLADYFCQQYNSYPRTF
GGGTKLEIKR.

MGTA-117 (AB85) is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells, and this antibody and others contemplated by the disclosure are described in U.S. Application Publication No. 20200407440 and/or PCT Application Publication No. WO2019084064. Epitope analysis of AB85 binding to CD177 is described in PCT Application Publication No. WO2020219770, which identified the following two epitopes within CD117:

KAEATNTGKYTCTNKHGLSNSIYVFVRDPA (SEQ ID NO: 17; amino acids 60-90), and RCPLTDPEVTNYSLKGCQGKP (SEQ ID NO: 18; amino acids 100-130).

The sequences of the variable heavy chain and variable light chains of AB85 are disclosed as SEQ ID NO: 143 and SEQ ID NO: 144 from PCT Application Publication No. WO2019084064, respectively.

The heavy chain variable region (VH) amino acid sequence of Ab85 is:

(SEQ ID NO: 19)
EVQLVQSGAEVKKPGESLKISCKGSGYSFTNYWIGWVRQMPGKGLEWMA
IINPRDSDTRYRPSFQGQVTISADKSISTAYLQWSSLKASDTAMYYCAR
HGRGYEGYEGAFDIWGQGTLVTVSS.

The VH CDR amino acid sequences of AB85 are as follows: NYWIG (SEQ ID NO: 20; VH CDR1); IINPRDSDTRYRPSFQG (SEQ ID NO: 21; VH CDR2); and HGRGYEGYEGAFDI (SEQ ID NO: 22; VH CDR3).

The light chain variable region (VL) amino acid sequence of AB85 is:

(SEQ ID NO: 23)
DIQMTQSPSSLSASVGDRVTITCRSSQGIRSDLGWYQQKPGKAPKLLIY
DASNLETGVPSRFSGSGSGTDFTLTISSLQPEDFATYYCQQANGFPLTF
GGGTKVEIK.

The VL CDR amino acid sequences of AB85 are as follows: RSSQGIRSDLG (SEQ ID NO: 24; VL CDR1); DASNLET (SEQ ID NO: 25; VL CDR2); and QQANGFPLT (SEQ ID NO: 26; VL CDR3).

FSI-174 is an anti-cKIT antibody being developed in combination with 5F9 as a non-toxic transplant conditioning regimen, as well as a treatment for targeted hematologic malignancies. The sequences of FSI-174 are disclosed in PCT Application Publication No. 2020/112687, U.S. Patent Application Publication No. 20200165337, and U.S. Pat. No. 11,041,022. In particular embodiments, an anti-c-kit antibody comprises the three CDRs or variable heavy chain regions present in any of AH1, AH2, AH3, AH4, or AH5 disclosed therein, and/or the three CDRs or variable heavy chain regions present in any of AL1 or AL2 disclosed therein.

In certain embodiments, the CDRs present in FSI-174 and related antibodies are as follows: VH CDR1=SYNMH (SEQ ID NO: 27); VH CDR2=VIYSGNGDTSY(A/N)QKF(K/Q)G (SEQ ID NO: 28); VH CDR3=ERDTRFGN (SEQ ID NO: 29); VL CDR1=RAS(D/E)SVDIYG(N/Q)SFMH (SEQ ID NO: 30); VL CDR2=LASNLES (SEQ ID NO: 31); and VL CDR3=QQNNEDPYT (SEQ ID NO: 32). A/N and the like indicate that the amino acid position may be either of the two amino acids, in this example, A or N. In certain embodiments, CDRs present in the heavy variable region are CDRs H1, H2 and H3 as defined by Kabat: H1=SYNMH (SEQ ID NO: 27); H2=VIYSGNGDTSYAQKFKG (SEQ ID NO: 33); H3=ERDTRFGN (SEQ ID NO: 29); and the CDRs present in the light variable region are CDRs L1, L2 and L3 as defined by Kabat: L1=RASESVDIYGQSFMH (SEQ ID NO: 34); L2=LASNLES (SEQ ID NO: 31); and L3=QQNNEDPYT (SEQ ID NO: 32), respectively except that 1, 2, or 3 CDR residue substitutions is/are present selected from N to A at heavy chain position 60, K to Q at heavy chain position 64 and N to Q at light chain position 30, positions being numbered according to Kabat. In certain embodiments, the antibody comprises any of the heavy chain variable region sequences (AH2, AH3, AH4) and/or light chain variable chain region sequences provided below (AL2), or the CDRs therein shown underlined:

AH2:
(SEQ ID NO: 35)
QVQLVQSGAEVKKPGASVKVSCKASGYTFTSYMNHWVRQAPGQGLEWMG
VIYSGNGDTSYAQKFKGRVTITADKSTSTAYMELSSLRSEDTAVYYCAR
ERDTREGNWGQGTLVTVSS
AH3:
(SEQ ID NO: 36)
QVQLVQSGAEVKKPGASVKVSCKASGYTFTSYMNHWVRQAPGQGLEWMG
VIYSGNGDTSYNQKFQGRVTITADKSTSTAYMELSSLRSEDTAVYYCAR
ERDTRFGNWGQGTLVTVSS
AH4
(SEQ ID NO: 37)
QVQLVQSGAEVKKPGASVKVSCKASGYTFTSYMNHWVRQAPGQGLEWMG
VIYSGNGDTSYAQKFQGRVTITADKSTSTAYMELSSLRSEDTAVYYCAR
ERDTREGNWGQGTLVTVSS
AL2:
(SEQ ID NO: 38)
DIVMTQSPLSLPVTPGEPASISCRASESVDIYGQSFMHWYQQKPGQPPK
LLIYLASNLESGVPDRFSGSGSGTDFTLKISRVEAEDVGVYYCQQNNED
PYTFGGGTKVEIK.

In certain embodiments, the anti-c-kit antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-c-kit antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-c-kit antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-c-kit antibody comprises a heavy chain or variable region thereof comprising one, two, or three heavy chain CDRs disclosed herein, e.g., a JSP191 heavy chain. In particular embodiments, the anti-c-kit antibody comprises a light chain or variable region thereof comprising one, two, or three light chain CDRs disclosed herein, e.g., a JSP191 light chain.

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization.

MGTA-117 is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells and this antibody and others contemplated by the disclosure are described in US 20200407440.

FSI-174 is an anti-c-kit antibody being develop in combination with 5F9 as a non-toxic transplant conditioning regimen, as well as a treatment for targeted hematologic malignancies.

In particular embodiments, the antibody may include one or more CDR with at least 70%, 80%, 90%, 95%, or 99% amino acid or nucleotide sequence identity to a CDR present in a humanized monoclonal antibody that binds c-kit, e.g., an antibody derived from any of the mouse antibodies SR1, ACK2, ACK4, 2B8, 3C11, MR-1, and CD122. In some embodiments, the antibody blocks the binding of stem cell factor (SCF) to stem cell factor receptor (CD117). Illustrative embodiments of anti-c-kit antibodies that may be used include JSP191, as well as those described in WO2007127317A2 and US20200165337A1, both incorporated herein in their entirety.

In certain embodiments, the anti-c-kit antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-c-kit antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-c-kit antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-c-kit antibody comprises a heavy chain or variable region thereof comprising one, two, or three heavy chain CDRs disclosed herein, e.g., a JSP191 heavy chain. In particular embodiments, the anti-c-kit antibody comprises a light chain or variable region thereof comprising one, two, or three light chain CDRs disclosed herein, e.g., a JSP191 light chain.

### II. Subjects

In certain embodiments, a subject selected for an anti-c-kit antibody treatment as described herein has or retains at least some healthy hematopoietic cells (HSC) to benefit from the selective depletion of diseased HSC. In some embodiments, a subject selected for an anti-c-kit treatment as described herein has at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% healthy hematopoietic stem cells (HSCs) as a percentage of total HSCs in the bone marrow prior to treatment. In particular embodiments, the percental of healthy HSCs increases over a course of treatment.

In some embodiments, the percentage of healthy hematopoietic stems cells in a subject, prior to administration of an anti-c-kit treatment as described herein, is assessed through a bone marrow biopsy, a blood count, or a blood film.

The treatments described herein have been shown to be effective in myelodysplastic syndrome (MDS) and acute myeloid leukemia (AML). However, these treatments are not intended to be limiting to these disease populations alone. Compositions and methods disclosed herein may be used to treat other disorders for which selective depletion of diseased HSC is indicated. Relevant diseases or disorders may include but are not limited to: acute myeloid leukemia (AML), chronic myeloid leukemia (CML), chronic myelomonocytic leukemia (CMML), acute lymphoblastic leukemia (ALL), hodgkin lymphoma, non-hodgkin lymphoma, clonal hematopoiesis of indeterminate potential (CHIP), clonal cytopenia of undetermined significance (CCUS) myelodysplastic syndromes (MDS), idiopathic cytopenia of undetermined significance (ICUS), and myeloproliferative neoplasms (MPN).

In some embodiments, the disease is a blood cancer, optionally a leukemia, a lymphoma, or a myelodysplastic syndrome (MDS). In some embodiments, the disease or disorder is multiple myeloma, chronic myelogenous leukemia (CML) myelodysplastic syndromes (MDS), a myeloproliferative neoplasm, or a myeloid leukemia, e.g., acute myeloid leukemia (AML) or chronic myeloid leukemia (CML). In some embodiments, the disease is MDS or AML. In some embodiments, the cancer is a lymphoid leukemia, e.g., acute lymphocytic leukemia (ALL) or chronic lymphocytic leukemia (CLL).

In some embodiments, the cancer is a myelodysplastic/myeloproliferative neoplasm (MDS/MPN), such as, e.g., chronic myelomonocytic leukemia (CMML). MDS/MPN have both “dysplastic” and “proliferative” features that cannot be classified as either myelodysplastic syndromes (MDS) or myeloproliferative neoplasms (MPN), and for this reason have been categorized as an overlap syndrome with its own distinct characteristics (MDS/MPN). CMML is cancer of the blood. CMML is considered to be one of the myelodysplastic/myeloproliferative neoplasms (MDS/MPN), a type of chronic blood cancer in which a person's bone marrow does not make blood effectively.

In some embodiments, the subject has a hematopoietic cell transplant comorbidity index (HCT-CI) greater than or equal to 3 (Sorror ML, et al. Hematopoietic cell transplantation (HCT)-specific comorbidity index: a new tool for risk assessment before allogeneic HCT. Blood. 2005;106(8):2912-2919.). In some embodiments, the subject has a hematopoietic cell transplant comorbidity index (HCT-CI) less than or equal to 3.

In some embodiments, the disease or disorder is multiple myeloma, severe combined immune deficiency (SCID), chronic myelogenous leukemia (CML), myelodysplastic syndromes (MDS), a myeloproliferative neoplasm, or acute myeloid leukemia (AML).

In certain embodiments, the disease treated according to the disclosure is referred to as MDS/AML, which includes both MDS and AML. Myelodysplastic syndromes (MDS) and acute myeloid leukemia (AML) exist along a continuous disease spectrum starting with early-stage MDS, which may progress to advanced MDS, AML, cured AML or resistant AML. The disease is characterized by an overproduction of immature blood cells. The resulting lack of mature, healthy blood cells causes anemia and an increased risk for infection and bleeding. Around 5-10% of patients with solid tumors who are treated with chemotherapy, radiation or autologous stem cell transplantation develop treatment-related MDS or AML.

Myelodysplastic syndromes (MDS) are a group of hematopoietic neoplasms characterized by abnormal differentiation and cytomorphology (i.e., dysplasia) of pluripotent hematopoietic progenitor cells (i.e., stem cells) residing in the myeloid compartment of the bone marrow (BM). These abnormalities lead to ineffective hematopoiesis and to cytopenia (i.e., lower-than-normal peripheral blood cell counts) of one or more lineages of the myeloid progenitor cells that manifests as anemia, neutropenia, and/or thrombocytopenia. Methods disclosed herein may be used to treat various forms of MDS, including but not limited to those shown in Table 1 below, which is reproduced from Chung, US Pharm. 2021;46(9):39-44. In certain embodiments, the methods result in decreased cytopenia.

TABLE 1
2016 WHO Classification of MDS
MDS Entities

MDS with single-lineage dysplasia (MDS-SLD)
MDS with multilineage dysplasia (MDS-SLD)
MDS with ring sideroblasts (MDS-RS): ≥15% ring sideroblasts in BM
or ≥5% with SF3B1 mutation
MDS with ring sideroblasts and single-lineage dysplasia (MDS-RS-SLD)
MDS with ring sideroblasts and multilineage dysplasia (MDS-RS-SLD)
MDS with excess blasts-1 (MDS-EB1): blasts in blood 2%-4%, blasts in
BM 5%-9%
MDS with excess blasts-2 (MDS-EB2): blasts in blood 5%-19%, blasts in
BM 10%-19%
MDS with isolated 5q-
MDS, unclassifiable (MDS-U)
BM: bone marrow: MDS: myelodysplastic syndromes: WHO: World Health Organization, Source: Reference 2.

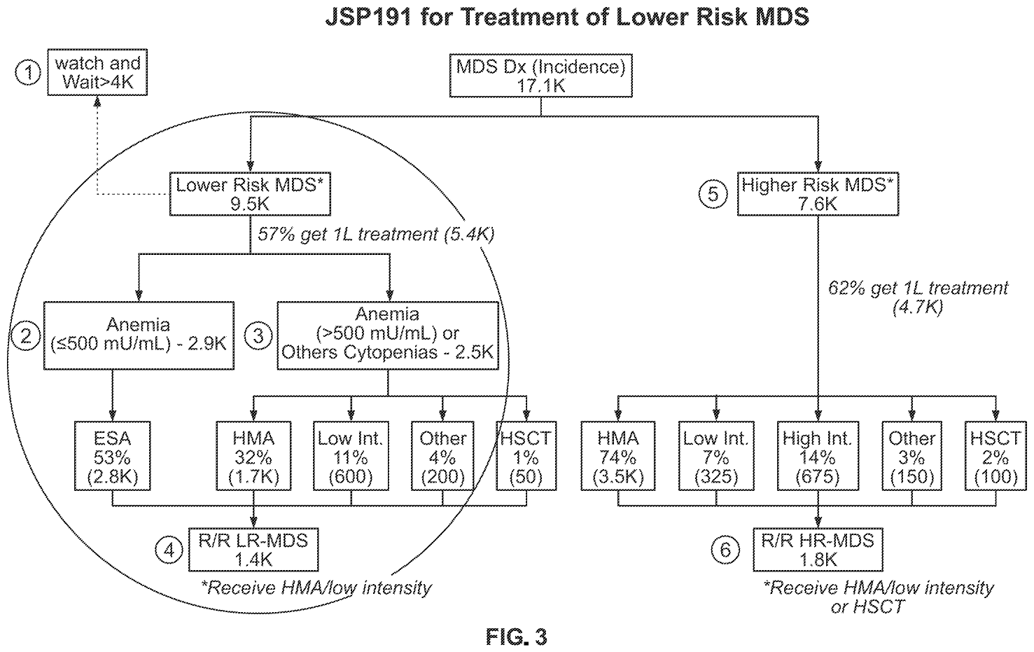

In some embodiments, a subject selected for an anti-c-kit treatment of MDS as described herein qualifies according to IPSS-R guidelines (e.g., see FIG. 3 and Greenberg et al., Revised international prognostic scoring system for myelodysplastic syndromes, Blood, Sep 20;120(12):2454-65; 2012.) as having lower risk myelodysplastic syndrome (MDS). In some embodiments, a subject selected for an anti-c-kit treatment as described herein qualifies according to IPSS-R guidelines as having medium risk MDS. In some embodiments, a subject selected for an anti-c-kit treatment as described herein qualifies according to IPSSR guidelines as having higher risk MDS. In particular embodiments, lower risk MDS patients are defined as those having a risk of very low, low, or intermediate disease according to the IPSS-R with a score ≤3.5 points, and higher-risk patients are those with intermediate risk and >3.5 points, high, or very high risk according to this scoring system. In certain embodiments, a subject has red blood cell transfusion dependence, thrombocytopenia, and/or neutropenia.

In some embodiments, the patient, e.g., a patient with CML, MDS, or AML, exhibits minimal identifiable disease (MID) and/or measurable residual disease (MRD), which may be detected by techniques including but not limited to cytogenetics, flow cytometry, and/or next-generation sequencing (NGS).

In some embodiments, administration of the combined therapies described herein reduces or rids the patient of minimal residual disease (MRD).

In some embodiments, a subject selected for an anti-c-kit treatment as described herein failed to respond to erythropoietin stimulating agents (ESA). In some embodiments the ESA is erythropoietin. In some embodiments the ESA is darbepoetin.

In some embodiments, a subject selected for an anti-c-kit treatment as described herein failed to respond to a chemotherapy. In some embodiments the chemotherapy is azacytidine, lenalidomide, decitabine, and/or luspatercept.

### III. Dosage and Administration

According to embodiments of methods of the disclosure, diseased HSC are selectively depleted at lower doses and dosing frequencies relative to healthy HSC. Thus, in particular embodiments, the anti-c-kit treatments described herein are administered in dosing regimens that preferentially deplete diseased HSCs relative to healthy cells, providing for healthy cell dominance and hematopoiesis in the bone marrow. Thus, the methods disclosed herein may be used to preferentially deplete diseased HSCs (as opposed to healthy HSCs) from a subject.

In some embodiments, a subject in need thereof is administered an anti-c-kit antibody in a dose of about 0.01 mg/kg to about 20 mg/kg. In some embodiments, the anti-c-kit antibody is any of SR-1, JSP191, MGTA-117, FSI-174, CDX-0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112 administered in a dose of about 0.01 mg/kg to about 20 mg/kg subject body weight. In some embodiments, the anti-c-kit antibody is administered in a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.25 mg/kg, about 2.5 mg/kg, about 2.75 mg/kg, about 3.0 mg/kg, about 3.25 mg/kg, about 3.5 mg/kg, about 3.75 mg/kg, about 4.0 mg/kg, about 4.5 mg/kg, about 5.0 mg/kg, about 5.5 mg/kg, or about 6.0 mg/kg. In certain embodiments, the anti-c-Kit antibody is administered at a dose from about 0.1 mg/kg to about 4 mg/kg, or about 0.5 mg/kg to about 4.0 mg/kg, or about 0.5 mg/kg to about 2.0 mg/kg. In particular embodiments, JSP191 is administered at any of these dosages. In particular embodiments, CDX0158 or CDX0159 is administered at any of these dosages. In particular embodiments, FSI-174 is administered at any of these dosages.

In some embodiments, a subject in need thereof is administered a single dose of an anti-c-kit antibody as described herein. In some embodiments the subject is administered two or more doses of the anti-c-kit antibody.

In some embodiments, a subject is administered an anti-c-kit antibody in a dosing regimen of about every week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, or about every 20 weeks, or about once per month, once every two months, or once every three months. In some embodiments, the subject is administered a dosing regimen of an anti-c-kit antibody wherein the time interval between each dose varies, e.g., a subject may be administered a dose about every week for about 4 weeks, followed by a dose about every 2 weeks for about 8 weeks, and combinations thereof. In one embodiment, a subject is administered an anti-c-kit antibody about once every eight weeks for four cycles, e.g., JSP191 at about 0.3 mg/kg, 0.6 mg/kg, 0.9 mg/kg, or 1.2 mg.kg body weight. In certain embodiments, the anti-c-kit antibody is JSP191 and is administered at a dose of from about 0.1 mg/kg to about 4 mg/kg, or about 0.5 mg/kg to about 4.0 mg/kg, or about 0.5 mg/kg to about 2.0 mg/kg, about 3.0 mg/kg, about 3.25 mg/kg, about 3.5 mg/kg, about 3.75 mg/kg, about 4.0 mg/kg, about 4.5 mg/kg, about 5.0 mg/kg, about 5.5 mg/kg, or about 6.0 mg/kg of the subject's body weight. In certain embodiments, JSP191 is administered to an MDS (e.g., low risk MDS) or AML patient at a dose of from about 0.1 mg/kg to about 4 mg/kg, or about 0.5 mg/kg to about 4.0 mg/kg, or about 0.5 mg/kg to about 2.0 mg/kg, about 3.0 mg/kg, about 3.25 mg/kg, about 3.5 mg/kg, about 3.75 mg/kg, about 4.0 mg/kg, about 4.5 mg/kg, about 5.0 mg/kg, about 5.5 mg/kg, or about 6.0 mg/kg of the subject's body weight about once a month for about four months.

In some embodiments a subject is administered an anti-c-kit antibody in a dosing regimen of about every month, about every 6 months, about every year, about every 2 years, about every 3 years, or about every 5 years. In some embodiments, the subject is administered an initial or loading dose of the anti-c-kit antibody as part of the dosing regimen,

In some embodiments, the dosing regimen is continued for about or at least a month, about or at least 6 months, about or at least a year, about or at least 2 years, about or at least 3 years, or about or at least 5 years. In some embodiments, the dosing regimen is continued until the subject is disease-free.

In some embodiments, an anti-c-kit antibody treatment as described herein increases the percentage of healthy hematopoietic stem cells (HSC) relative to total HSC in a subject's bone marrow to at least 50%, at least 60%, at least 70%, at least 80%, at least 90%, at least 95%, at least 99%, or at least 100% of all HSCs present in the subject's bone marrow.

In some embodiments, the anti-c-kit antibody treatment is associated with long term dominance of healthy hematopoietic stem cells (HSCs) in the subject's bone marrow. As used herein “long term dominance” of healthy HSC may refer to a prolonged increase in percentage of healthy HSC in the bone marrow, as assessed after any period of time following treatment, e.g., 6 months, 1 year, 2 years, 3 years, 4 years, 5 years, 6 years, 7 years, 8 years, 9 years, or 10 years after treatment.

In some embodiments, the percentage of healthy hematopoietic stems cells after administration of an anti-c-kit treatment is assessed through a bone marrow biopsy, a blood count, or a blood film from the subject.

According to embodiments of the methods disclosed herein, monotherapy treatment with the anti-c-kit antibody is sufficient, and the subject does not need to be treated with another therapeutic agent for the disease being treated. In other embodiments, the subject is treated with the anti-c-kit antibody is combination with another chemotherapeutic agent or immunotherapy for treatment of the disease being treated.

In some embodiments, the subject administered an anti-c-kit treatment as described herein does not receive a hematopoietic cell transplant (HCT) within at least 4 months, at least 6 months, at least 1 year, at least 2 years, or for any period following administration of the anti-c-kit antibody.

In some embodiments, the subject administered an anti-c-kit treatment as described herein does not receive total body irradiation (TBI) within at least 4 months, at least 6 months, at least 1 year, at least 2 years, or for any period following administration of the anti-c-kit antibody.

In some embodiments, the subject administered an anti-c-kit treatment as described herein does not receive a chemotherapy including but not limited to azacytidine, lenalidomide, decitabine, and/or luspatercept within at least 4 months, at least 6 months, at least 1 year, or at least 2 years, or for any period following administration of the anti-c-kit antibody.

In some embodiments, an anti-c-kit treatment as described herein is associated with reduced side effects relative to other treatments including but not limited to hematopoietic cell transplant (HCT), total body irradiation (TBI), and/or chemotherapy. Measurement of fewer side effects may be determined by the number of patient complaints, patient/doctor visits, and/or hospitalizations following treatment. In some embodiments, the reduced side effects comprise lower risk of hematologic toxicity and/or lower risk of mortality by infection. In some embodiments, the methods disclosed herein result in reduced cytopenia as compared to other treatments, such as, e.g., HCT.

In some embodiments, an anti-c-kit antibody as described herein may be administered as a combination therapy comprising an anti-CD47 antibody.

In some embodiments, and anti-c-kit antibody as described herein may be administered as a combination therapy comprising a chemotherapeutic agent, e.g., azacytidine, lenalidomide, decitabine, and/or luspatercept. In particular embodiments, the anti-c-kit antibody, e.g., JSP-191 is administered to the subject in combination with azacytidine. The two agents may be administered at the same or different times, and in some embodiments, the two agents are both present in the subject for a time period.

In some embodiments, the anti-c-kit antibody therapy (or combination therapy) may be delivered orally, subcutaneously, intravenously, intranasally, transdermally, intraperitoneally, intramuscularly, intrapulmonary, vaginally, rectally, or intraocularly. In some embodiments, the anti-c-kit antibody, e.g., JSP191, is administered systemically, e.g., intravenously or subcutaneously. In some embodiments, an anti-c-kit antibody is delivered directly to the bone marrow.

### IV. Pharmaceutical Compositions

In some embodiments, the anti-c-kit antibody (or combination therapy) is present in a pharmaceutical composition. In particular embodiments, the pharmaceutical compositions may be in a water-soluble form, such as in pharmaceutically acceptable salts, which is meant to include both acid and base addition salts. Pharmaceutically acceptable acid addition salts include but are not limited to: hydrochloric acid, hydrobromic acid, sulfuric acid, nitric acid, phosphoric acid, organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid. Pharmaceutically acceptable base addition salts include those derived from inorganic bases such as sodium, potassium, lithium, ammonium, calcium, magnesium, iron, zinc, copper, manganese, aluminum salts and the like. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Pharmaceutical compositions as described herein may also include one or more of the following: carrier proteins such as serum albumin; buffers; fillers such as microcrystalline cellulose, lactose, corn and other starches; binding agents; and polyethylene glycol.

The compositions for administration will commonly include the antibody dissolved in a pharmaceutically acceptable carrier, preferably an aqueous carrier. A variety of aqueous carriers can be used, e.g., buffered saline. The composition may contain pharmaceutically acceptable auxiliary substances as required to approximate physiological conditions such as pH and buffering agents, toxicity countering agents, e.g., sodium acetate, sodium chloride, potassium chloride, calcium chloride, and sodium lactate. The concentration of active agents in the formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

### V. Kits

The disclosure further provides kits comprising an anti-c-kit antibody as described herein, e.g., a kit for administration of the anti-c-kit antibody by a medical professional or the subject in need thereof. The kit may comprise for example, a container, and one or more doses of the anti-c-kit antibody, and instructions for use thereof. In certain embodiments, the kit comprises multiple doses of the anti-c-kit antibody, suitable for a dosing regimen over a period of time. In certain embodiments, each dose comprises the same amount of the anti-c-kit antibody, while in other embodiments, the kit may comprises two or more different doses of the anti-c-kit antibody. In certain embodiments, each dose in present within a separate container. In particular embodiments, each of the one or more doses comprises about 0.5 mg to about 800 mg of the anti-c-kit antibody, optionally about 5 mg to about 800 mg of the anti-c-kit antibody. In particular embodiments, one or more doses comprises about 40 mg to about 50 mg, optionally about 45 mg, of the anti-c-kit antibody. In particular embodiments, one or more doses comprises about 140 mg to about 160 mg, optionally about 150 mg, of the anti-c-kit antibody.

### EXAMPLES

### Example 1

### JSP191 as a Primary Treatment for MDS and AML

A clinical study was conducted to demonstrate that treatment of myelodysplastic syndrome (MDS) and acute myeloid leukemia (AML) patients with the anti-c-kit antibody, JSP191, was effective in depleting diseased stem and progenitor cells.

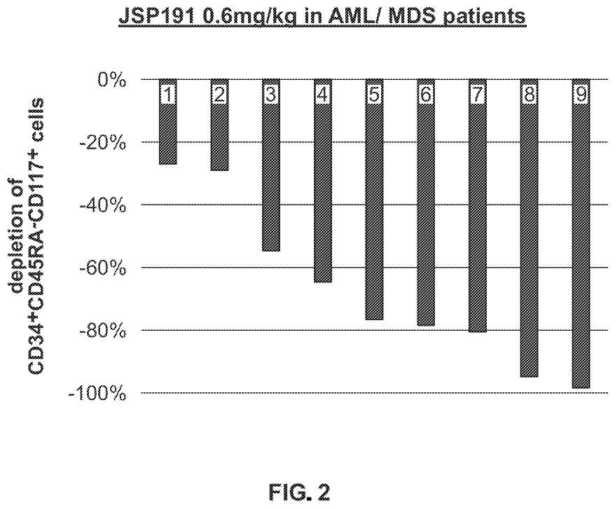

Nine patients having either higher risk MDS or AML were treated with a single dose of 0.6 mg/kg JSP191 administered intravenously. Notably, JSP191 depleted 30-95% of diseased HSC cells (CD34+CD45RA-CD117+ HSCs) at 10-14 days post-treatment (FIG. 2). Furthermore, JSP191 administered intravenously at 0.6 mg/kg was safe and well-tolerated in this ongoing clinical trial. No significant JSP191-related infusion reactions were observed, and no significant adverse events were observed.

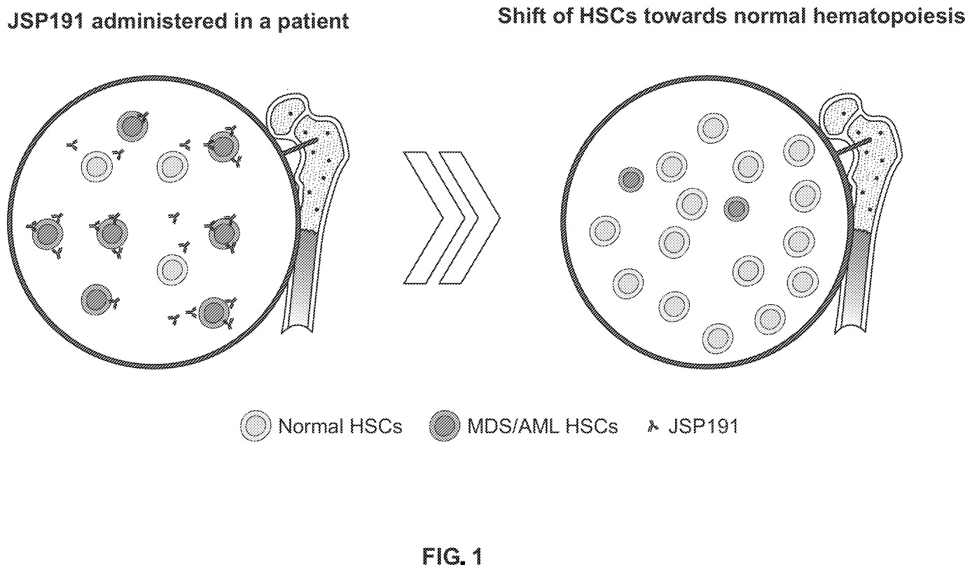

These studies demonstrate that for patients who retain at least a small proportion of normal HSCs, treatment with an anti-c-kit antibody can serve as a primary treatment to deplete diseased HSCs and allow replacement of the diseased HSCs through hematopoiesis of healthy HSCs, as diagrammed in FIG. 1.

### Example 2

### JSP191 as a Second Line Therapy in Patients With Lower Risk MDS

A phase 1 clinical study is conducted to demonstrate that treatment of low-risk myelodysplastic syndrome (MDS) patients with the anti-c-kit antibody, JSP191, is effective in depleting diseased stem and progenitor cells.

Four cohorts of MDS patients are treated with various doses of JSP191, including 0.3 mg/kg (w/ sentinel dose of 0.1 mg/kg), 0.6 mg/kg, 0.9 mg/kg, and 12 mg/kg. The MDS patients are dosed every 8 weeks×4 cycles. Each cohort includes 3-6 patients. The patients have IPSS-R very low, low or intermediate risk MDS, i.e., lower risk MDS patients. The patients have RBC transfusion dependence, thrombocytopenia, or neutropenia.

The studies are expected to show safety and tolerability of JSP191 at these doses, as well as efficacy by HI-E/HI-P/HI-N and duration of response, reduction in RBC transfusions, ORR, and duration of response by IWG 2006, and progression free survival. Hematological improvements may be analyzed and demonstrated as described in Okamura, et al., Leuk Res Rep. 2014; 3(1): 24-27. Treatment with JSP191 is expected to cause a depletion of diseased HSCs, followed by recovery and expansion of healthy HSCs.

These studies are expected to demonstrate that for lower risk MDS patients who retain at least a small proportion of normal HSCs, treatment with an anti-c-kit antibody can serve as a primary treatment to deplete diseased HSCs and allow replacement of the diseased HSCs through hematopoiesis of healthy HSCs, as diagrammed in FIG. 1.

The various embodiments described above can be combined to provide further embodiments.

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

All of the U.S. patents, U.S. patent application publications, U.S. patent application, foreign patents, foreign patent application and non-patent publications referred to in this specification and/or listed in the Application Data Sheet are incorporated herein by reference, in their entireties.

1. A method of treating a hematopoietic stem cell (HSC) disease or disorder in a subject, optionally myelodysplastic syndrome (MDS) or acute myeloid leukemia (AML), the method comprising administering to the subject an anti-c-kit antibody in a dose effective to achieve long term presence or dominance of healthy HSCs in the subject's bone marrow.

2. The method of claim 1, wherein the method selectively depletes diseased HSCs as compared to healthy HSCs.

3. The method of claim 1 or 2, wherein the anti-c-kit antibody comprises one or more complementarity-determining regions (CDRs) present in a monoclonal antibody selected from the group consisting of: SR-1, JSP191, MGTA-117, FSI-174, CDX-0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112.

4. The method of any one of claims 1-3, wherein the anti-c-kit antibody comprises one or more complementarity-determining regions (CDRs) present in a humanized version of a monoclonal antibody selected from the group consisting of: ACK2, ACK4, 2B8, 3C11, MR-1, and CD122.

5. The method of any one of claims 1-4, wherein the anti-c-kit antibody comprises the CDRs of an antibody that blocks the binding of stem cell factor (SCF) to stem cell factor receptor (CD117), optionally wherein the antibody is JSP191.

6. The method of any one of claims 1-5, wherein the subject is administered about 0.01 mg/kg to about 10 mg/kg of the anti-c-kit antibody, optionally about 0.1 mg/kg to about 10 mg/kg of the anti-c-kit antibody.

7. The method of any one of claims 1-5, wherein the subject is administered about 0.6 mg/kg of the anti-c-kit antibody.

8. The method of any one of claims 1-5, wherein the subject is administered about 2 mg/kg of the anti-c-kit antibody.

9. The method of any one of claims 1-8, wherein the subject's bone marrow comprises at least 1%, at least 2%, at least 3%, at least 4%, at least 5%, at least 6%, at least 7%, at least 8%, at least 9%, at least 10%, at least 15%, or at least 20% healthy HSCs prior to treatment.

10. The method of any one of claims 1-9, wherein the disease or disorder is a myelodysplastic syndrome (MDS).

11. The method of any one of claims 1-10, wherein the MDS is a lower risk MDS.

12. The method of any one of claims 1-10, wherein the disease or disorder is acute myeloid leukemia (AML).

13. The method of any one of claims 1-12, wherein the anti-c-kit antibody is delivered to the subject systemically, optionally wherein the anti-c-kit antibody is delivered intravenously or subcutaneously.

14. The method of any one of claims 1-12, wherein the anti-c-kit antibody is delivered to the subject locally, optionally wherein the anti-c-kit antibody is delivered directly to the bone marrow.

15. The method of any one of claims 1-14, wherein the subject is administered two or more doses of the anti-c-kit antibody.

16. The method of claim 15, wherein one of the two or more doses is administered every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks, optionally wherein the time interval between dosages varies.

17. The method of any one of claims 1-16, wherein the subject has failed an erythropoiesis-stimulating agent (ESA) therapy, optionally wherein the ESA therapy comprises Erythropoietin and/or Darbepoetin.

18. The method of any one of claims 1-17, wherein the method has reduced side effects as compared to treatment with a chemotherapeutic agent, optionally wherein the chemotherapeutic agent comprises Azacytidine, Lenalidomide, Decitabine, and/or Luspatercept.

19. The method of claim 18, wherein the reduced side effects are lower risk of hematologic toxicity and lower risk of mortality by infection.

20. The method of any one of claims 1-19, wherein the method results in an increase in the percentage of normal HSCs in the bone marrow to at least 50%, at least 60%, at least 70%, at least 80%, at least 90%, at least 95%, at least 99%, or at least 100% of all HSCs.

21. The method of any one of claims 1-20, wherein the subject does not have a hematopoietic cell transplant (HCT) within at least 4 months, at least 6 months, at least 1 year, or at least 2 years following administration of the anti-c-kit antibody.

22. The method of any one of claims 1-21, wherein the anti-c-kit antibody is administered in combination with an anti-CD47 antibody.

23. The method of any one of claims 1-22, wherein the anti-c-kit antibody is administered in combination with a chemotherapeutic agent.

24. The method of claim 23, wherein the chemotherapeutic agent is azacitidine.

25. A method of depleting endogenous diseased hematopoietic stem cells (HSCs) in a subject, comprising administering to the subject an anti-c-kit antibody, wherein the method results in an increase in the percentage of healthy hematopoietic stem cells (HSCs) present in the subject's bone marrow.

26. The method of claim 25, wherein the anti-c-kit antibody comprises one or more complementarity-determining regions (CDRs) present in a monoclonal antibody selected from the group consisting of: SR-1, JSP191, MGTA-117, FSI-174, CDX0158, CDX0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112, optionally JSP191, CDX0158, CDX0159, or FSI-174.

27. The method of claim 25, wherein the anti-c-kit antibody comprises one or more complementarity-determining regions (CDRs) present in a humanized version of a monoclonal antibody selected from the group consisting of: ACK2, ACK4, 2B8, 3C11, MR-1, and CD122.

28. The method of claim 25, wherein the anti-c-kit antibody comprises the CDRs of an antibody
that blocks the binding of stem cell factor (SCF) to stem cell factor receptor (CD117), optionally wherein the antibody is JSP191.

29. The method of any one of claims 25-28, wherein the subject is administered about 0.01 mg/kg to about 20 mg/kg of the anti-c-kit antibody, optionally about 0.1 mg/kg to about 10 mg/kg of the anti-c-kit antibody.

30. The method of any one of claims 25-28, wherein the subject is administered about 0.6 mg/kg of the anti-c-kit antibody.

31. The method of any one of claims 25-28, wherein the subject is administered about 2 mg/kg of the anti-c-kit antibody.

32. The method of claim 1, wherein the subject's bone marrow comprises at least 1%, at least 2%, at least 3%, at least 4%, at least 5%, at least 6%, at least 7%, at least 8%, at least 9%, at least 10%, at least 15%, or at least 20% healthy HSCs prior to treatment.

33. The method of any one of claims 25-32, wherein is used to treat a disease or disorder selected from the group consisting of: acute myeloid leukemia (AML), chronic myeloid leukemia (CML), chronic myelomonocytic leukemia (CMML), acute lymphoblastic leukemia (ALL), hodgkin lymphoma, non-hodgkin lymphoma, clonal hematopoiesis of indeterminate potential (CHIP), clonal cytopenia of undetermined significance (CCUS) myelodysplastic syndromes (MDS), idiopathic cytopenia of undetermined significance (ICUS), and myeloproliferative neoplasms (MPN).

34. The method of claim 33, wherein the disease or disorder is an MDS.

35. The method of claim 34, wherein the MDS is a lower risk MDS.

36. The method of any one of claims 25-35, wherein the anti-c-kit antibody is delivered to the subject systemically, optionally wherein the anti-c-kit antibody is delivered intravenously or subcutaneously.

37. The method of any one of claims 25-35, wherein the anti-c-kit antibody is delivered to the subject locally, optionally wherein the anti-c-kit antibody is delivered directly to the bone marrow.

38. The method of any one of claims 25-37, wherein the subject is administered two or more doses of the anti-c-kit antibody.

39. The method of claim 38, wherein one of the two or more doses is administered about every week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, or about every 12 weeks, optionally wherein the time interval between each dose varies.

40. The method of any one of claims 25-39, wherein the subject has failed an erythropoiesis-stimulating agent (ESA) therapy, optionally wherein the ESA therapy comprises Erythropoietin and/or Darbepoetin.

41. The method of any one of claims 25-40, wherein the method has reduced side effects as compared to treatment with a chemotherapeutic agent, optionally wherein the chemotherapeutic agent comprises Azacytidine, Lenalidomide, Decitabine, and/or Luspatercept.

42. The method of claim 41, wherein the reduced side effects comprise lower risk of hematologic toxicity and lower risk of mortality by infection.

43. The method of any one of claims 25-42, wherein the method results in an increase in the percentage of normal HSCs in the bone marrow to at least 50%, at least 60%, at least 70%, at least 80%, at least 90%, at least 95%, at least 99%, or at least 100% of all HSCs, and optionally where the method results in long term dominance of healthy hematopoietic stem cells (HSCs) in the subject's bone marrow.

44. The method of any one of claims 25-43, wherein the subject does not have a hematopoietic cell transplant (HCT) within at least 4 months, at least 6 months, at least 1 year, or at least 2 years following administration of the anti-c-kit antibody.

45. A kit comprising one or more doses of an anti-c-kit antibody.

46. The kit of claim 45, wherein each of the one or more doses comprises about 0.5 mg to about 800 mg of the anti-c-kit antibody, optionally about 5 mg to about 800 mg of the anti-c-kit antibody.

47. The kit of claim 46, wherein the kit comprises one or more doses comprising about 40 mg to about 50 mg, optionally about 45 mg, of the anti-c-kit antibody.

48. The kit of claim 46, wherein the kit comprises one or more doses comprising about 140 mg to about 160 mg, optionally about 150 mg, of the anti-c-kit antibody.


SEQUENCE LISTING (38 sequences)

SEQ ID NO: 1 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 2 - protein, 466 aa, Homo sapiens
MDWTWRVFCLLAVAPGAHSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 238 aa, Homo sapiens
MVLQTQVFISLLLWISGAYGDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Homo sapiens
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 5 - protein, 111 aa, Homo sapiens
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 6 - protein, 4 aa, Homo sapiens
YNMH

SEQ ID NO: 7 - protein, 16 aa, Homo sapiens
IYSGNGDTSYNQKFKG

SEQ ID NO: 8 - protein, 8 aa, Homo sapiens
ERDTRFGN

SEQ ID NO: 9 - protein, 15 aa, Homo sapiens
RASESVDIYGNSFMH

SEQ ID NO: 10 - protein, 7 aa, Homo sapiens
LASNLES

SEQ ID NO: 11 - protein, 9 aa, Homo sapiens
QQNNEDPYT

SEQ ID NO: 12 - protein, 107 aa, synthetic construct
  VARIANT 10: note = X is an amino acid with an aromatic or aliphatic hydroxyl side chain
  VARIANT 46: note = X is an amino acid with an aliphatic or aliphatic hydroxyl side chain
  VARIANT 63: note = X is an amino acid with an aliphatic hydroxyl side chain
  VARIANT 80: note = X is an amino acid with an aliphatic hydroxyl side chain or is P
  VARIANT 85: note = X is an amino acid with a charged or acidic side chain
  VARIANT 87: note = X is an amino acid with an aromatic side chain
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 13 - protein, 117 aa, synthetic construct
  VARIANT 11: note = X is an amino acid with an aliphatic side chain
  VARIANT 20: note = X is an amino acid with an aliphatic side chain
  VARIANT 39: note = X is an amino acid with a polar or basic side chain
  VARIANT 69: note = X is an amino acid with an aliphatic side chain
  VARIANT 71: note = X is an amino acid with an aliphatic side chain
  VARIANT 74: note = X is an amino acid with an acidic side chain
  VARIANT 83: note = X is an amino acid with an acidic or amide derivative side chain
  VARIANT 92: note = X is an amino acid with an aliphatic hydroxyl side chain
QVQLVQSGAEXKKPGASVKXSCKASGYTFTDYYINAVVXQAPGKGLEWIARIYPGSGNTYYNEKFKGRXTXTAXKSTSTAYMXLSSLRSEDXAVYFCARGVYYFDYWGQGTTVTVSS

SEQ ID NO: 14 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSS

SEQ ID NO: 15 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSA

SEQ ID NO: 16 - protein, 108 aa, synthetic construct
DIVMTQSQKFMSTSVGDRVSVTCKASQNVRTNVAWYQQKPGQSPKALIYSASYRYSGVPDRFTGSGSGTDFTLTISNVQSEDLADYFCQQYNSYPRTFGGGTKLEIKR

SEQ ID NO: 17 - protein, 31 aa, Homo sapiens
EKAEATNTGKYTCTNKHGLSNSIYVFVRDPA

SEQ ID NO: 18 - protein, 21 aa, Homo sapiens
RCPLTDPEVTNYSLKGCQGKP

SEQ ID NO: 19 - protein, 123 aa, synthetic construct
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 20 - protein, 5 aa, synthetic construct
NYWIG

SEQ ID NO: 21 - protein, 17 aa, synthetic construct
IINPRDSDTRYRPSFQG

SEQ ID NO: 22 - protein, 14 aa, synthetic construct
HGRGYEGYEGAFDI

SEQ ID NO: 23 - protein, 107 aa, synthetic construct
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 24 - protein, 11 aa, synthetic construct
RSSQGIRSDLG

SEQ ID NO: 25 - protein, 7 aa, synthetic construct
DASNLET

SEQ ID NO: 26 - protein, 9 aa, synthetic construct
QQANGFPLT

SEQ ID NO: 27 - protein, 5 aa, synthetic construct
SYNMH

SEQ ID NO: 28 - protein, 17 aa, synthetic construct
  VARIANT 12: note = X is A or N
  VARIANT 16: note = X is K or Q
VIYSGNGDTSYXQKFXG

SEQ ID NO: 29 - protein, 8 aa, synthetic construct
ERDTRFGN

SEQ ID NO: 30 - protein, 15 aa, synthetic construct
  VARIANT 4: note = X is D or E
  VARIANT 11: note = X is N or Q
RASXSVDIYGXSFMH

SEQ ID NO: 31 - protein, 7 aa, synthetic construct
LASNLES

SEQ ID NO: 32 - protein, 9 aa, synthetic construct
QQNNEDPYT

SEQ ID NO: 33 - protein, 17 aa, synthetic construct
VIYSGNGDTSYAQKFKG

SEQ ID NO: 34 - protein, 15 aa, synthetic construct
RASESVDIYGQSFMH

SEQ ID NO: 35 - protein, 117 aa, synthetic construct
QVQLVQSGAEVKKPGASVKVSCKASGYTFTSYMNHWVRQAPGQGLEWMGVIYSGNGDTSYAQKFKGRVTITADKSTSTAYMELSSLRSEDTAVYYCARERDTRFGNWGQGTLVTVSS

SEQ ID NO: 36 - protein, 117 aa, synthetic construct
QVQLVQSGAEVKKPGASVKVSCKASGYTFTSYMNHWVRQAPGQGLEWMGVIYSGNGDTSYNQKFQGRVTITADKSTSTAYMELSSLRSEDTAVYYCARERDTRFGNWGQGTLVTVSS

SEQ ID NO: 37 - protein, 117 aa, synthetic construct
QVQLVQSGAEVKKPGASVKVSCKASGYTFTSYMNHWVRQAPGQGLEWMGVIYSGNGDTSYAQKFQGRVTITADKSTSTAYMELSSLRSEDTAVYYCARERDTRFGNWGQGTLVTVSS

SEQ ID NO: 38 - protein, 111 aa, synthetic construct
DIVMTQSPLSLPVTPGEPASISCRASESVDIYGQSFMHWYQQKPGQPPKLLIYLASNLESGVPDRFSGSGSGTDFTLKISRVEAEDVGVYYCQQNNEDPYTFGGGTKVEIK